(19) United States
(12) Patent Application Publication
(10) Pub. No.: US 2024/0077555 A1
(43) Pub. Date: Mar. 7, 2024

(54) SUPERCONDUCTING MAGNET AND MRI APPARATUS

(71) Applicant: CANON MEDICAL SYSTEMS CORPORATION, Otawara-shi (JP)

(72) Inventors: Takehiro SHIBUYA, Nasushiobara (JP); Hiromi KAWAMOTO, Yaita (JP)

(73) Assignee: CANON MEDICAL SYSTEMS CORPORATION, Otawara-shi (JP)

(21) Appl. No.: 18/451,279

(22) Filed: Aug. 17, 2023

(30) Foreign Application Priority Data
Sep. 1, 2022 (JP) 2022-139080

(51) Int. Cl. G01R 33/3815; H01F 6/00; H01F 6/02; H01F 6/06

(52) U.S. Cl. CPC G01R 33/3815; H01F 6/008; H01F 6/02; H01F 6/06

(57) ABSTRACT

In one embodiment, a superconducting magnet includes: at least one primary superconducting coil configured to generate a primary static magnetic field by a persistent current flowing during a persistent current mode; at least one secondary superconducting coil configured to generate a secondary static magnetic field different from the primary static magnetic field in response to external control; and a static-magnetic-field control switch configured to (i) supply the secondary superconducting coil with part of the persistent current to generate the secondary static magnetic field by being closed in response to the external control during the persistent current mode and (ii) stop energization of the secondary superconducting coil and generation of the secondary static magnetic field by being opened in response to the external control during the persistent current mode.

### CROSS-REFERENCE TO RELATED APPLICATION

This application is based upon and claims the benefit of priority from Japanese Patent Application No. 2022-139080, filed on Sep. 1, 2022, the entire contents of which are incorporated herein by reference.

### TECHNICAL FIELD

Disclosed embodiments relate to a superconducting magnet and a magnetic resonance imaging (MRI) apparatus.

### BACKGROUND

An MRI apparatus is an imaging apparatus that magnetically excites nuclear spin of an object placed in a static magnetic field by applying a radio frequency (RF) signal having the Larmor frequency and reconstructs an image on the basis of magnetic resonance (MR) signals emitted from the object due to the excitation.

An MRI apparatus includes a static magnetic field magnet for generating a static magnetic field. In particular, in an MRI apparatus installed in a medical institution such as a hospital for examination and diagnosis, a considerably strong static magnetic field is required, and thus, a superconducting magnet is used.

In a static magnetic field magnet using a superconducting magnet, a superconducting coil is cooled down to an extremely low temperature by, for example, liquid helium. This static magnetic field magnet generates a static magnetic field by applying an electric current supplied from a static-magnetic-field power supply to the superconducting coil in an excitation mode. Afterward, when the static magnetic field magnet shifts to a persistent current mode, the static-magnetic-field power supply is disconnected.

Among imaging methods using the MRI apparatus, an imaging method called a pre-polarization is known. In the pre-polarization, a static magnetic field (i.e., pre-polarization field) of predetermined strength for aligning the spin axes of protons in an object is applied to the object for, e.g., several seconds before imaging the object. Then, part of an imaging pulse sequence is applied under the state where the pre-polarization field is instantaneously shifted to zero. Afterward, the pre-polarization field is returned to the predetermined strength, and this cycle is repeated to acquire MR signals required for image reconstruction.

In this manner, in imaging using the pre-polarization method, the operation of instantaneously increasing and decreasing the strength of the pre-polarization field (i.e., static magnetic field) is performed. In the conventional technology, this pre-polarization field is generated by using, for example, a normal conducting coil. However, it is known that increasing the strength of the pre-polarization field increases the SNR (signal to noise ratio).

Thus, if the pre-polarization field can be generated by using a superconducting coil, an image with a high SNR can be obtained. In this case, it is necessary to instantaneously increase and decrease the strength of the static magnetic field to be generated by the superconducting coil.

However, if the strength of the static magnetic field is increased or decreased instantaneously by increasing or decreasing the electric current flowing through the superconducting coil, at least the following two problems may arise.

The first problem is that a so-called AC loss is generated by increasing or decreasing the electric current flowing through the superconducting coil and the heat ascribable to this AC loss causes a risk of quench.

The second problem is that the rapid change (dI/dt) in electric current flowing through the superconducting coil generates a high voltage (=L·(dI/dt)) due to an inductance component (L) of the superconducting coil.

Not only in the above-described pre-polarization but also in conventional imaging methods, there is a demand to change static magnetic field strength and/or static magnetic field distribution in a short time even after shifting to the persistent current mode.

Conventionally, in case of emergency (for example, when a magnetic material is brought into an examination room where the superconducting magnet is installed because of some reason), an emergency shutdown device is activated to forcibly shift the superconducting magnet to the quenched state and demagnetizing it. However, once the superconducting magnet shifts to the quenched state, it takes a long time and many efforts to return the superconducting magnet to the original imageable state.

### BRIEF DESCRIPTION OF THE DRAWINGS

In the accompanying drawings:

FIG. 1 is a block diagram illustrating an overall configuration of an MRI apparatus according to the first embodiment;

FIG. 2A is a schematic view of a superconducting magnet according to the first embodiment as viewed from the direction along the central axis of the cylindrical shape;

FIG. 2B is a cross-sectional view of the superconducting magnet taken along the line Y-Y′ in FIG. 2A for illustrating its internal configuration;

FIG. 3 is an equivalent circuit diagram illustrating electrical connection of the superconducting magnet according to the first embodiment;

FIG. 4A to FIG. 4C are schematic diagrams illustrating the concepts of respective three operation modes of the superconducting magnet;

FIG. 5A and FIG. 5B are schematic diagrams illustrating static magnetic field distribution A in a persistent current mode;

FIG. 6A and FIG. 6B are schematic diagrams illustrating static magnetic field distribution B in a static-magnetic-field control mode;

FIG. 7A to FIG. 7H are timing charts illustrating a pulse sequence in the pre-polarization using the superconducting magnet of the first embodiment;

FIG. 8A and FIG. 8B are schematic diagrams illustrating a configuration of the superconducting magnet according to a modification of the first embodiment;

FIG. 9 is a schematic diagram illustrating a first configuration of the superconducting magnet according to the second embodiment;

FIG. 10 is a schematic diagram illustrating a second configuration of the superconducting magnet according to the second embodiment;

FIG. 11A is a schematic view of the superconducting magnet according to the second embodiment as viewed from the direction along the central axis of the cylindrical shape; and

FIG. 11B is a cross-sectional view of the superconducting magnet taken along the line X-X′ in FIG. 11A for illustrating its internal configuration.

### DETAILED DESCRIPTION

Hereinbelow, embodiments of the present invention will be described by referring to the accompanying drawings.

In one embodiment, a superconducting magnet includes: at least one primary superconducting coil configured to generate a primary static magnetic field by a persistent current flowing during a persistent current mode; at least one secondary superconducting coil configured to generate a secondary static magnetic field different from the primary static magnetic field in response to external control; and a static-magnetic-field control switch configured to (i) supply the secondary superconducting coil with part of the persistent current to generate the secondary static magnetic field by being closed in response to the external control during the persistent current mode and (ii) stop energization of the secondary superconducting coil and generation of the secondary static magnetic field by being opened in response to the external control during the persistent current mode.

(MRI Apparatus)

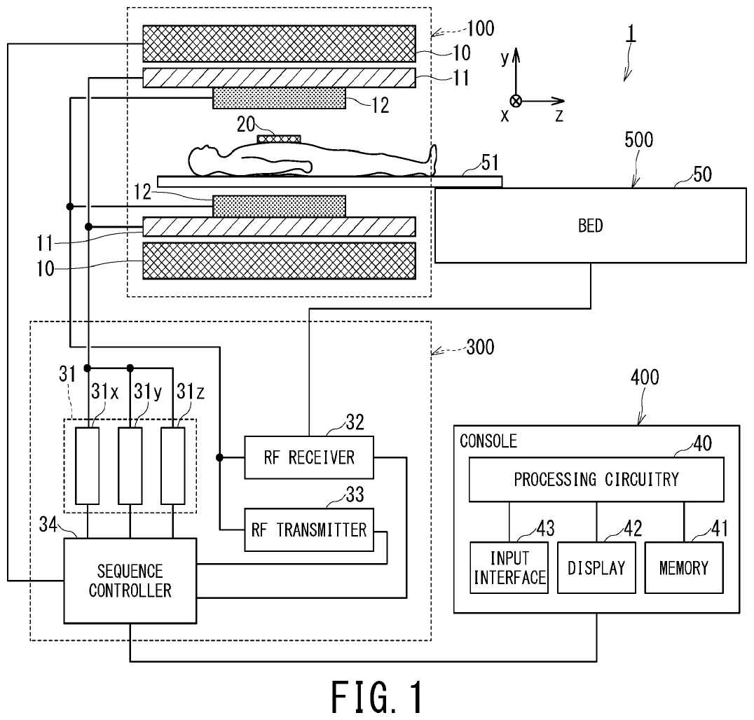

FIG. 1 is a block diagram illustrating an overall configuration of an MRI apparatus 1 provided with a superconducting magnet 10 according to the first embodiment. The MRI apparatus 1 includes: a gantry 100; a bed 500; a control cabinet 300; and a console 400, for example.

The gantry 100 includes hardware components such as the superconducting magnet 10, a gradient coil 11, and a WB (Whole Body) coil 12, and these components are housed in a cylindrical housing. The bed 500 includes a bed body 50 and a table 51. The MRI apparatus 1 also includes at least one local coil 20 (i.e., surface coil 20) disposed close to an object.

The control cabinet 300 includes: three gradient coil power supplies 31 (31x for an X-axis, 31y for a Y-axis, and 31z for a Z-axis); an RF receiver 32; an RF transmitter 33; and a sequence controller 34.

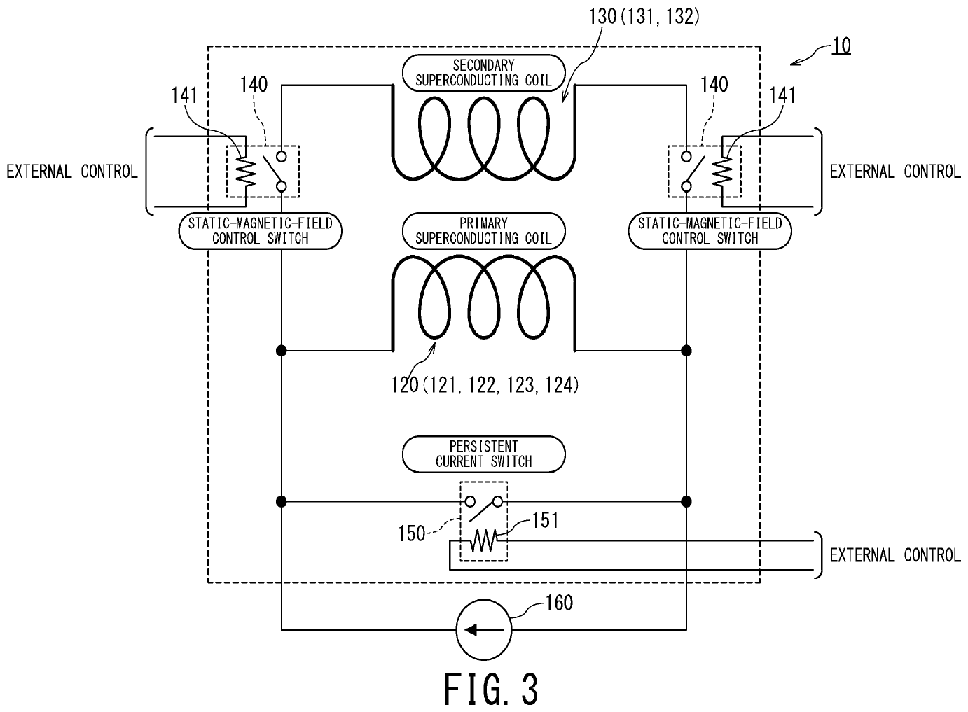

The superconducting magnet 10 of the gantry 100 is substantially in the form of a cylinder and generates a static magnetic field inside a bore, which is a space inside the cylindrical structure of the superconducting magnet 10 and is also an imaging region of an object such as human body or a patient. The superconducting magnet 10 includes at least one superconducting coil inside, and the superconducting coil is cooled down to an extremely low temperature by liquid helium. The superconducting magnet 10 generates a static magnetic field by applying an electric current provided from a static-magnetic-field power supply (160 in FIG. 3) to the superconducting coil in an excitation mode. Afterward, the superconducting magnet 10 shifts to a persistent current mode, and then the static-magnetic-field power supply is disconnected. Once it enters the persistent current mode, the superconducting magnet 10 continues to generate a strong static magnetic field for a long time, for example, over one year. The configuration and function of the superconducting magnet 10 according to the embodiment will be described below in detail.

The gradient coil 11 is also substantially in the form of a cylinder and is fixed to the inside of the superconducting magnet 10. This gradient coil 11 applies gradient magnetic fields to the object in the respective directions of the X-axis, the Y-axis, and the Z-axis by using electric currents supplied from the respective gradient coil power supplies 31x, 31y, and 31z.

The bed body 50 of the bed 500 can move the table 51 in the vertical direction, and moves the table 51 with the object placed thereon to a predetermined height before imaging. Afterward, during time of imaging, the bed body 50 moves the table 51 in the horizontal direction so as to move the object to the inside of the bore.

The WB coil 12 is substantially formed into a cylindrical shape and fixed to the inside of the gradient coil 11 so as to surround the object. The WB coil 12 applies RF pulses transmitted from the RF transmitter 33 to the object, and receives MR signals emitted from the object due to excitation of hydrogen nuclei.

The local coil 20 is also called a surface coil or an RF coil that receives MR signals emitted from the object at a position close to the body surface of the object. The local coil 20 includes a plurality of coil elements, for example. There are various local coils 20 adaptable to the anatomical imaging part of the object, such as the head, the chest, the spine, the lower limbs, and the whole body. FIG. 1 illustrates the local coil 20 for imaging the chest.

The RF transmitter 33 transmits each RF pulse to the WB coil 12 on the basis of an instruction from the sequence controller 34. The RF receiver 32 receives MR signals detected by the WB coil 12 and/or the local coil 20, and transmits raw data obtained by digitizing the detected MR signals to the sequence controller 34.

The sequence controller 34 performs a scan of the object by driving the gradient coil power supplies 31, the RF transmitter 33, and the RF receiver 32 under the control of the console 400. The sequence controller 34 receives the raw data acquired by the scan from the RF receiver 32 and then transmits the raw data to the console 400.

The sequence controller 34 includes processing circuitry (not shown). This processing circuitry is configured as hardware such as a processor configured to execute predetermined programs, a field programmable gate array (FPGA), and an application specific integrated circuit (ASIC), for example.

The console 400 is configured as a computer that includes processing circuitry 40, a memory 41, an input interface 43, and a display 42.

The memory 41 is a recording medium including a read-only memory (ROM) and a random access memory (RAM) in addition to an external memory device such as a hard disk drive (HDD) and an optical disc device. The memory 41 stores various programs to be executed by a processor of the processing circuitry 40 in addition to various data and information.

The input interface 43 includes various devices for a user to input various data and information, and is configured as, for example, a mouse, a keyboard, a trackball, and/or a touch panel.

The display 42 is a display device such as a liquid crystal display panel, a plasma display panel, and an organic EL panel.

The processing circuitry 40 is, for example, a circuit provided with a CPU and/or a special-purpose or general-purpose processor. The processor implements various functions described below by executing programs stored in the memory 41. The processing circuitry 40 may be configured with hardware such as an FPGA and an ASIC. The processing circuitry 40 can implement various functions by combining hardware processing and software processing based on its processor and programs.

The console 400 controls the entirety of the MRI apparatus 1. Specifically, the console 400 receives various instructions and information such as imaging conditions that are inputted by a user such as a medical imaging technologist through the mouse and/or the keyboard of the input interface 43. The processing circuitry 40 causes the sequence controller 34 to perform a scan on the basis of the inputted imaging conditions, and reconstructs images based on the raw data transmitted from the sequence controller 34. The reconstructed images are displayed on the display 42 or stored in the memory 41.

(Superconducting Magnet of First Embodiment)

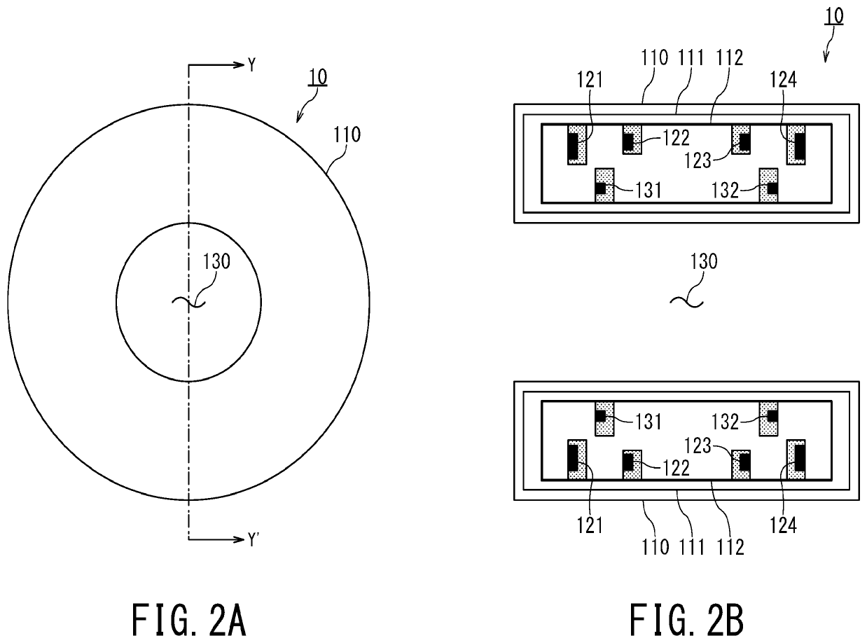

FIG. 2A is a schematic view of the superconducting magnet 10 according to the first embodiment as viewed from the direction along the central axis of its cylindrical shape. FIG. 2B is a cross-sectional view of the superconducting magnet 10 taken along the line Y-Y′ in FIG. 2A for illustrating its internal configuration.

As shown in FIG. 2A and FIG. 2B, the superconducting magnet 10 of the gantry 100 has a substantially cylindrical shape, and a bore 130 is formed as a cylindrical imaging space along the central axis of the cylindrical shape.

The superconducting magnet 10 includes at least one primary superconducting coil 120 and at least one secondary superconducting coil 130. As described below, the primary superconducting coil 120 generates a primary static magnetic field by using a persistent current that flows during the persistent current mode. The secondary superconducting coil 130 generates a secondary static magnetic field different from the primary static magnetic field in response to external control.

In the aspect shown in FIG. 2A and FIG. 2B, the superconducting magnet 10 includes four primary superconducting coils 121, 122, 123 and 124, and two secondary superconducting coils 131 and 132. Hereinafter, the four primary superconducting coils 121, 122, 123 and 124 are collectively referred to as the primary superconducting coil 120, and the two secondary superconducting coils 131 and 132 are collectively referred to as the secondary superconducting coil 130.

Each of the primary and secondary superconducting coils 120 and 130 is formed by using an ultra-fine multifilamentary wire structure in which a superconducting material such as niobium titanium (Nb—Ti) is made into many thin filaments and embedded in a normal-conduction base material such as copper. Additionally or alternatively, each of the primary and secondary superconducting coils 120 and 130 is formed by using a rare-earth-based or bismuth-based high-temperature superconducting wire in a tape shape, for example.

The primary and secondary superconducting coils 120 and 130 are submerged in a liquid helium vessel 112 filled with liquid helium, for example. The entire liquid helium vessel 112 is surrounded by a vacuum vessel 110 called a cryostat that prevents heat from entering. Between the liquid helium vessel 112 and the vacuum vessel 110, a heat-radiation shield-plate 111 made of aluminum is provided, for example.

FIG. 3 is an equivalent circuit diagram illustrating electrical connection of the superconducting magnet 10. The superconducting magnet 10 includes the primary superconducting coil 120, the secondary superconducting coil 130, two static-magnetic-field control switches 140, and a persistent current switch 150. The superconducting magnet 10 is connectable with a static-magnetic-field power supply 160 that applies an electric current to the superconducting magnet 10 in the excitation mode.

The persistent current switch 150 is connected in parallel to the static-magnetic-field power supply 160 and is also connected in parallel to the primary superconducting coil 120. The persistent current switch 150 is composed of a superconducting member and a heater 151 disposed close to this superconducting member. When the heater 151 is turned off, the superconducting member is cooled by the liquid helium and thus is maintained in a superconducting state. In other words, when the heater 151 is turned off, the persistent current switch 150 is closed.

On the other hand, when the heater 151 is heated by external control, i.e., when the heater 151 is turned on, the superconducting member shifts to a normal conducting state, and thus the persistent current switch 150 is open.

One of the two static-magnetic-field control switches 140 is provided between one end of the primary superconducting coil 120 and one end of the secondary superconducting coil 130. The other of the two static-magnetic-field control switches 140 is provided between the other end of the primary superconducting coil 120 and the other end of the secondary superconducting coil 130.

Each static-magnetic-field control switch 140 is also composed of a superconducting member and a heater 141 disposed close to this superconducting member, similarly to the persistent current switch 150. When both heaters 141 are turned off, the superconducting members are cooled by liquid helium and thus is maintained in a superconducting state. In other words, when both heaters 141 are turned off, both static-magnetic-field control switches 140 are closed.

When both heaters 141 are heated by external control, i.e., when both heaters 141 are turned on, the superconducting members shifts to a normal conducting state and thus both static-magnetic-field control switches 140 are open.

Although one static-magnetic-field control switch 140 is respectively provided at both ends of the secondary superconducting coil 130 in FIG. 3, the superconducting magnet may be configured such that one static-magnetic-field control switch 140 is provided at only one end of the secondary superconducting coil 130.

Note that the superconducting member provided in the static-magnetic-field control switch 140 may be configured by non-inductively winding a superconducting wire.

By winding the superconducting member (i.e., superconducting wire) included in the static magnetic field control switch 140 in a non-inductive winding, influence of the magnetic field generated by the static magnetic field control switch 140 on the static magnetic field distribution to be formed by the first and second superconducting coils 120 and 130 can be suppressed.

The primary superconducting coil 120 is a superconducting coil that generates the primary static magnetic field by using a persistent current. Although FIG. 3 schematically illustrates one primary superconducting coil 120, the primary superconducting coil 120 may include a pair of primary superconducting coils 121 and 124 or include two pairs of primary superconducting coils 121, 122, 123, and 124 as illustrated in FIG. 2B, for example. The primary superconducting coil 120 may include more number of superconducting coils.

When the primary superconducting coil 120 includes a plurality of superconducting coils, all of the superconducting coils may be connected in series or in parallel, or a circuit configuration combining series connection and parallel connection may be used.

The secondary superconducting coil 130 generates a secondary static magnetic field by causing part of the persistent current flowing through the primary superconducting coil 120 to flow through the secondary superconducting coil 130 under external control. Although FIG. 3 schematically illustrates one primary superconducting coil 130 similarly to the primary superconducting coil 120, the secondary superconducting coil 130 may include a pair of secondary superconducting coils 131 and 132 as illustrated in FIG. 2B or may include only one superconducting coil. Additionally or alternatively, the secondary superconducting coil 130 may include three or more superconducting coils.

The secondary superconducting coil 130 is preferably configured as a superconducting coil that is non-inductively wound with respect to the primary superconducting coil 120.

Non-inductive winding is, for example, a method of winding a wire for canceling or reducing combined inductance components (L components) of two coils by, for example, reversing the winding directions of two coils and reversing the directions of currents flowing through the respective coils.

As is well known, when there is change in electric current flowing through the superconducting coil, a back electromotive force (voltage) is generated in the direction that cancels this change. When the change (dI/dt) in electric current flowing through the superconducting coil is large, the inductance component (L) of the superconducting coil generates a high voltage (=L(dI/dt)) between both ends of the superconducting coil.

By contrast, in this embodiment, since the secondary superconducting coil 130 is non-inductively wound with respect to the primary superconducting coil 120, the combined inductance component (L component) of the two coils 120 and 130 is suppressed, and thus, generation of a high voltage is suppressed even if the electric currents flowing through the secondary superconducting coil 130 and the primary superconducting coil 120 change significantly.

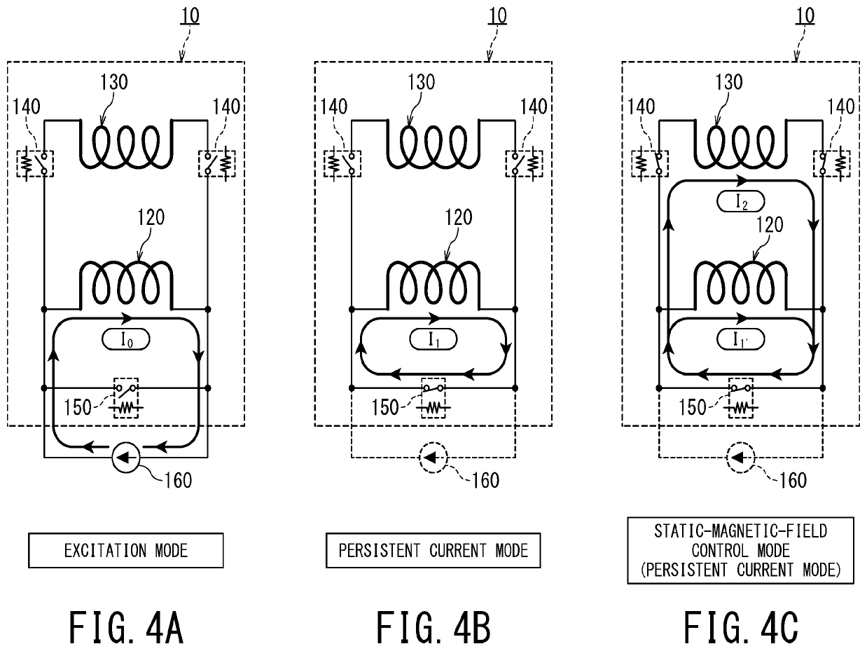

The operation of the superconducting magnet 10 having the above-described configuration will be described below by referring to FIG. 4A to FIG. 7H.

FIG. 4A to FIG. 4C are schematic diagrams illustrating the concepts of respective three operation modes of the superconducting magnet 10. FIG. 4A shows the operation in the excitation mode. The excitation mode is an operation mode in which the static magnetic field power supply 160 is externally connected to the superconducting magnet 10 and supplies current to the superconducting magnet 10, such that the superconducting magnet 10 that has not yet generated a static magnetic field (i.e., the non-operating superconducting magnet 10) starts to generate a static magnetic field of a predetermined strength.

Both the persistent current switch 150 and the static-magnetic-field control switches 140 are open in the excitation mode. In other words, the superconducting magnet 10 is controlled from the outside (for example, the sequence controller 34 and/or the operation unit (not shown) of the superconducting magnet 10) in such a manner that both the heater 151 of the persistent current switch 150 and the heaters 141 of the two static-magnetic-field control switch 140 are turned on.

In the excitation mode, the excitation current I₀ supplied from the static-magnetic-field power supply 160 flows through only the primary superconducting coil 120. The excitation current I₀ is gradually increased from zero, and when the excitation current I₀ reaches the rated value, the superconducting magnet 10 is shifted from the excitation mode to the persistent current mode.

FIG. 4B shows the operation in the persistent current mode. When the excitation current I₀ reaches the rated value, the heater 151 of the persistent current switch 150 is turned off by external control such as control from the sequence controller 34 based on user's operation, or and control from the operation unit of the superconducting magnet 10, for example. In response to turn-off of the heater 151, the superconducting member provided in the persistent current switch 150 is cooled down and transitions from the normal conducting state to the superconducting state, and the persistent current switch 150 is closed.

As a result, a persistent current loop is formed by the persistent current switch 150 and the primary superconducting coil 120, and a persistent current I₁ flows through the superconducting coil 120. Since both the primary superconducting coil 120 and the persistent current switch 150 are in the superconducting state and are zero in electrical resistance, the persistent current I₁ continues to flow through the persistent current loop even after the static-magnetic-field power supply 160 is disconnected from the superconducting magnet 10. This state is the persistent current mode.

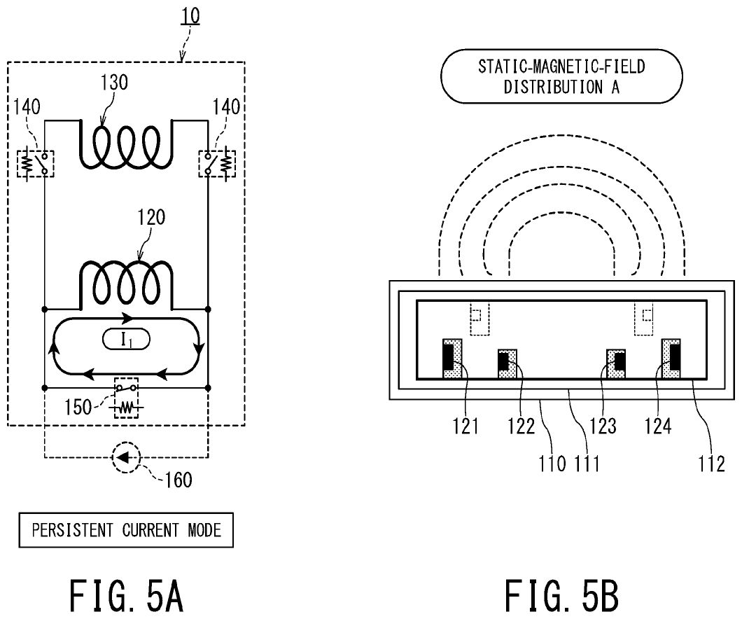

FIG. 5B is a schematic diagram illustrating static magnetic field distribution A in the persistent current mode. FIG. 5A is the same as FIG. 4B. The static magnetic field distribution A shown in FIG. 5B is not in a technically exact shape but represents the static magnetic field distribution generated in the persistent current mode has a specific shape, for example, the magnetic field strength is maximized near the center of the bore 130.

In the case of the conventional superconducting magnet, once it shifts to the persistent current mode, its static magnetic field strength and the shape of the static magnetic field distribution cannot be changed except for fine adjustment by shimming.

In contrast to the conventional superconducting magnet, the superconducting magnet 10 of the present embodiment is provided with the secondary superconducting coil 130 and the static-magnetic-field control switch 140. This configuration enables the superconducting magnet 10 to change strength and distribution shape of the static magnetic field without using an external power supply such as the static-magnetic-field power supply 160, even after shifting to the persistent current mode. Hereinafter, the operation mode of changing static magnetic field strength and the shape of static magnetic field distribution is referred to as a “static-magnetic-field control mode”. Since the external power supply, such as the static-magnetic-field power supply 160, is disconnected in the static-magnetic-field control mode, this operation mode can be interpreted as one of variations of the persistent current mode.

FIG. 4C shows the operation in the static-magnetic-field control mode. The shift from the persistent current mode in FIG. 4B to the static-magnetic-field control mode in FIG. 4C is achieved by turning off the heaters 141 of both static-magnetic-field control switches 140 under the external control, such as control from the sequence controller 34 based on the user's operation, or control from the operation unit of the superconducting magnet 10, for example.

Turning off the heaters 141 causes the superconducting members provided in the static-magnetic-field control switches 140 to be cooled down and transited from the normal conducting state to the superconducting state, and the static-magnetic-field control switches 140 to be closed.

As a result, part of the persistent current flowing through the primary superconducting coil 120 is branched, and a branch current I₂ corresponding to a predetermined branch ratio flows through the secondary superconducting coil 130. Since both the secondary superconducting coil 130 and the static-magnetic-field control switches 140 are in the superconducting state and are zero in electrical resistance, the branch current I₂ can continue to flow as the persistent current without attenuation.

The secondary static magnetic field is generated by the branch current I₂ flowing through the secondary superconducting coil 130. Although the electric current flowing through the primary superconducting coil 120 changes due to the branch current flowing into the secondary superconducting coil 130, the primary superconducting coil 120 continues to generate the primary static magnetic field by the changed persistent current I_{1′}. As a result, the superconducting magnet 10 as a whole generates a combined static magnetic field that combines the primary static magnetic field generated by the primary superconducting coil 120 and the secondary static magnetic field generated by the secondary superconducting coil 130.

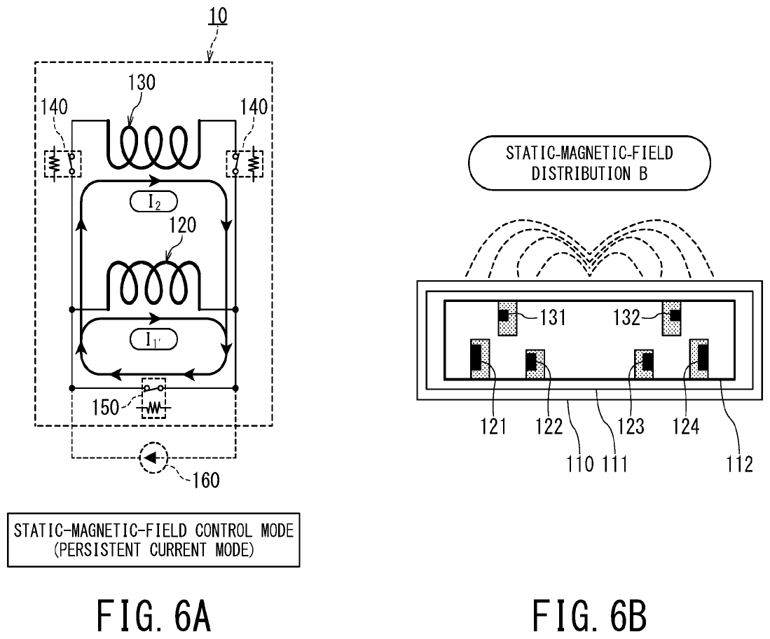

FIG. 6B is a schematic diagram illustrating static magnetic field distribution B (i.e., combined static magnetic field distribution) in the static-magnetic-field control mode. FIG. 6A is the same as FIG. 4C. The distribution shape of the secondary static magnetic field can be changed by adjusting the direction of the electric current flowing through the secondary superconducting coil 130, number of the plurality of superconducting coils constituting the secondary superconducting coil 130, spatial arrangement of the superconducting coils, and parameters of the superconducting coils such as diameter and current density, which enables generation of the combined static magnetic field distribution in a desired shape.

Further, instantaneous switching between the persistent current mode and the static-magnetic-field control mode can be achieved by switching the opening and closing of the static-magnetic-field control switches 140 in response to the external control, which enables instantaneous switching between the static magnetic field distribution in the persistent current mode (for example, the static magnetic field distribution A) and the combined static magnetic field distribution in the static-magnetic-field control mode (for example, the static magnetic field distribution B).

For example, strength of the combined static magnetic field in a predetermined region can be reduced or decreased to substantially zero by generating spatial distribution of the secondary static magnetic field that reduces or cancels the primary static magnetic field in the predetermined region. As a result, in the predetermined region, magnetic field strength can be instantaneously switched between the predetermined static magnetic field strength (i.e., the rated value of the static magnetic field strength) in the persistent current mode, reduced static magnetic field strength in the static-magnetic-field control mode, and substantially zero static magnetic field strength.

Further, for example, the combined magnetic field strength in the predetermined region can be adjusted by winding the secondary superconducting coil 130 in such a manner that the non-inductive winding of the secondary superconducting coil 130 does not completely cancel the magnetic field of the primary superconducting coil 120.

Note that, conventionally, in case of emergency (for example, when a magnetic material is for some reason brought into an examination room where the superconducting magnet is installed), an emergency shutdown device is activated to forcibly shift the superconducting magnet to the quenched state and demagnetize it, for example, However, once the superconducting magnet shifts to the quenched state, it takes a long time and many efforts to return the superconducting magnet to the original imageable state.

The superconducting magnet 10 of the present embodiment can instantaneously change the static magnetic field strength from the rated value to zero by being shifted from the persistent current mode to the static-magnetic-field control mode without being shifted to the quenched state, as described above. For example, when the user presses an emergency shutdown button provided in an emergency shutdown device, its emergency magnetic-field shutdown function is activated. Along with the activation of this emergency magnetic-field shutdown function, the secondary superconducting coil 130 generates the secondary static magnetic field in such a manner that, the primary static magnetic field is canceled by this secondary static magnetic field, and thus the strength of the combined magnetic field of these primary and secondary static magnetic fields is instantaneously reduced from the rated value to zero without bringing the primary superconducting coil 120 into the quenched state.

In addition, even after the emergency state is released, static magnetic field strength can be instantaneously returned from zero to the rated value.

The switching between the persistent current mode and the static-magnetic-field control mode significantly changes the electric currents flowing through the primary superconducting coil 120 and the secondary superconducting coil 130. Nevertheless, since the secondary superconducting coil 130 is non-inductively wound with respect to the primary superconducting coil 120 as described above, generation of a high voltage in the primary superconducting coil 120 and the secondary superconducting coil 130, due to the back electromotive force, can be suppressed.

Further, it is generally known that when alternating current or fluctuating current flows through a superconducting coil, a loss called an AC loss (or alternating current loss) occurs and this loss generates heat. If the AC loss is large and the resultant heat generation is large, quench may occur.

In the conventional superconducting magnet, when trying to rapidly decrease the static magnetic field from the rated value to the desired value or to zero, or rapidly increase the static magnetic field from zero to the desired value or to the rated value, the electric current of the superconducting coil needs to be rapidly changed between the rated current and the desired value or zero with the static magnetic field power supply being connected, which causes a large amplitude difference and a large AC loss.

Considering the above problem, in the superconducting magnet 10 of the present embodiment, strength of the primary static magnetic field can be changed from the rated value to a desired value or zero, by branching part (e.g., half) of the persistent current flowing through the primary superconducting coil 120 and applying an electric current for canceling the primary static magnetic field to the secondary superconducting coil 130. Thus, the current change of the primary superconducting coil 120 can be reduced (for example, halved) as compared with the conventional superconducting magnet. Hence, according to the superconducting magnet 10 configured as described above, the AC loss can be suppressed, and the risk of quench can be reduced.

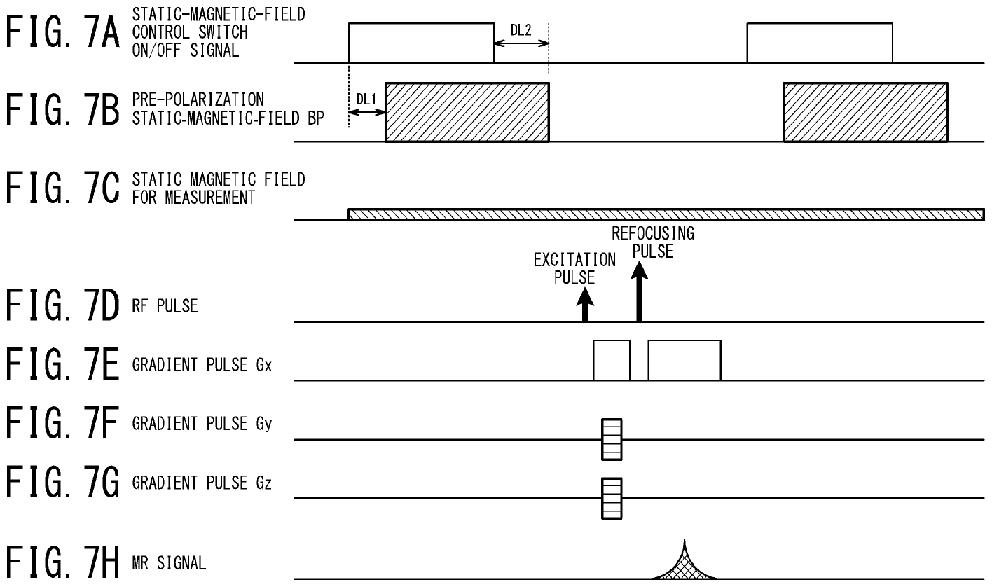

FIG. 7A to FIG. 7H are timing charts illustrating a pulse sequence in an imaging method called the pre-polarization, in which the superconducting magnet 10 according to the present embodiment is used. The pulse sequence of the pre-polarization is disclosed in, for example, Non-Patent Document 1 as follows.
[Non-Patent Document 1] A. N. Matlashov et al., “SQUID-based systems for co-registration of ultra-low field nuclear magnetic resonance images and magnetoencephalography”, Physica C 482 (2012) 19-26

In this imaging method as shown in FIG. 7B, a static magnetic field Bp (called a pre-polarization field) of predetermined strength for aligning the spin axes of the protons of the object is applied to the object for, e.g., several seconds before imaging the object, and then an imaging pulse sequence (for example, the pulse sequence shown in FIG. 7C to FIG. 7H) is applied while the pre-polarization field is instantaneously shifted to zero. Afterward, the pre-polarization field Bp is returned to the predetermined strength, and this cycle is repeated to acquire MR signals necessary for image reconstruction.

If the pre-polarization field Bp can be made to have large magnetic field strength by using a superconducting coil, an image with a high SNR can be obtained. Note that, in the pre-polarization, it is necessary to instantaneously increase or decrease the strength of the static magnetic field (Bp), which is generated by the superconducting coil.

As described above, the superconducting magnet 10 of the present embodiment can instantaneously increase or decrease static magnetic field strength by controlling the static-magnetic-field control switches 140 from the outside (FIG. 7A), and therefore, it is suitable for the imaging method using the above-described pulse sequence.

Note that there is a delay time DL1, which is attributable to heating of the heaters 141, from ON/OFF switching of the heater 141 of each static-magnetic-field control switch 140 by an external control signal until the superconducting member of each static-magnetic-field control switch 140 being transited from the superconducting state to the normal conducting state (i.e., until strength of the static magnetic field Bp switches from zero to a predetermined value). Similarly, there is a delay time DL2, which is attributable to cooling of the heaters 141, from ON/OFF switching of the heater 141 of each static-magnetic-field control switch 140 until the superconducting member of each static-magnetic-field control switch 140 being transited from the normal conducting state to the superconducting state (i.e., until strength of the static magnetic field Bp switches from the predetermined value to zero).

Thus, the static magnetic field can be switched in synchronization with a predetermined pulse sequence by determining the ON/OFF timing of the heater 141 of each static-magnetic-field control switch 140 in consideration of the above-described delay times DL1 and DL2.

The above-described delay times DL1 and DL2 may be determined by premeasurement. Additionally or alternatively, the delay times DL1 and DL2 determined in advance may be corrected by the temperature inside the superconducting magnet measured in real time.

### First Modification of First Embodiment

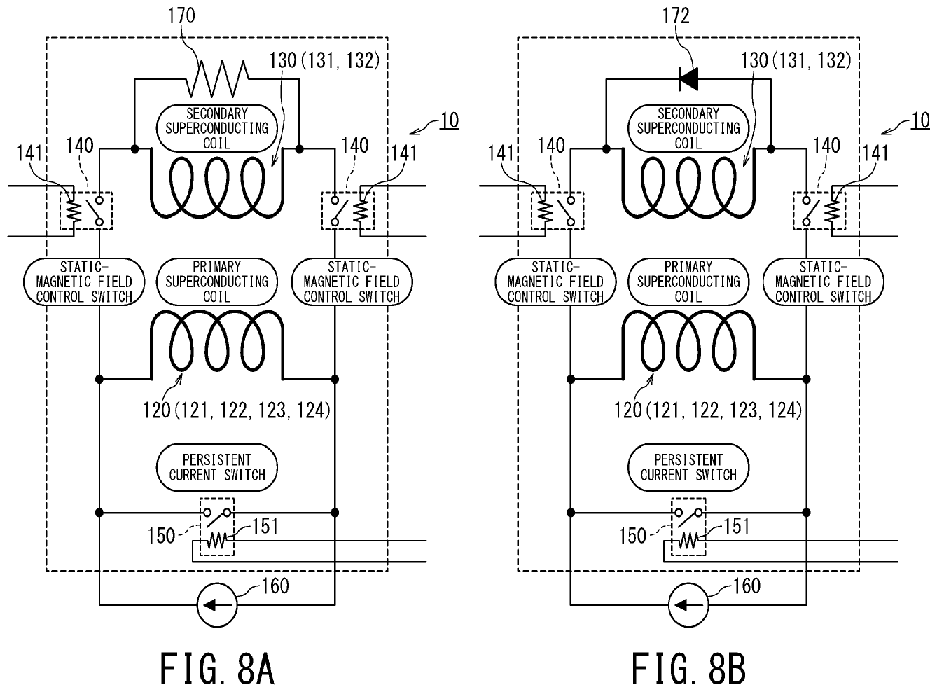

FIG. 8A and FIG. 8B are schematic diagrams illustrating a configuration of the superconducting magnet 10 according to a first modification of the first embodiment. In the first modification of the first embodiment, in order to suppress potential surge voltage that may occur when switching the static-magnetic-field control switches 140, or in order to give a predetermined time constant to the electric current flowing through the secondary superconducting coil 130 when increasing or decreasing the electric current, a resistor 170 and/or a diode 172 are provided in parallel with the secondary superconducting coil 130.

FIG. 8A illustrates a configuration in which the resistor 170 is provided in parallel with the secondary superconducting coil 130, and FIG. 8B illustrates a configuration in which the diode 172 is provided in parallel with the secondary superconducting coil 130.

### Second Modification of First Embodiment

The superconducting magnet 10 may be configured in such a manner that the secondary superconducting coil 130 and an external power supply (not shown) are always connected without the static-magnetic-field control switch 140 interposed inbetween, and the external power supply supplies an electric current to the secondary superconducting coil 130.

In the second modification, the external power supply, which is installed outside the vacuum vessel (cryostat) 110 in a normal temperature environment (for example, in a normal temperature environment of approximately 300K), and the secondary superconducting coil 130, which is installed in a cryogenic environment (for example, in an extremely low temperature environment of about 4K) inside the vacuum vessel 110, are always connected. Thus, the temperature difference between the external power supply and the secondary superconducting coil 130 becomes considerably large. As a result, heat enters the secondary superconducting coil 130 from the external power supply via the connection line, which may cause a problem that maintaining the superconducting state of the secondary superconducting coil 130 becomes difficult.

Hence, in the second modification, it is preferred to interpose a high-temperature superconducting connecting wire, which reaches the superconducting state at a temperature of about 50K, between the secondary superconducting coil 130 and the external power supply, for example. Such a configuration avoids direct thermal connection between the normal temperature environment outside and the cryogenic environment inside the vacuum vessel 110.

(Superconducting Magnet of Second Embodiment)

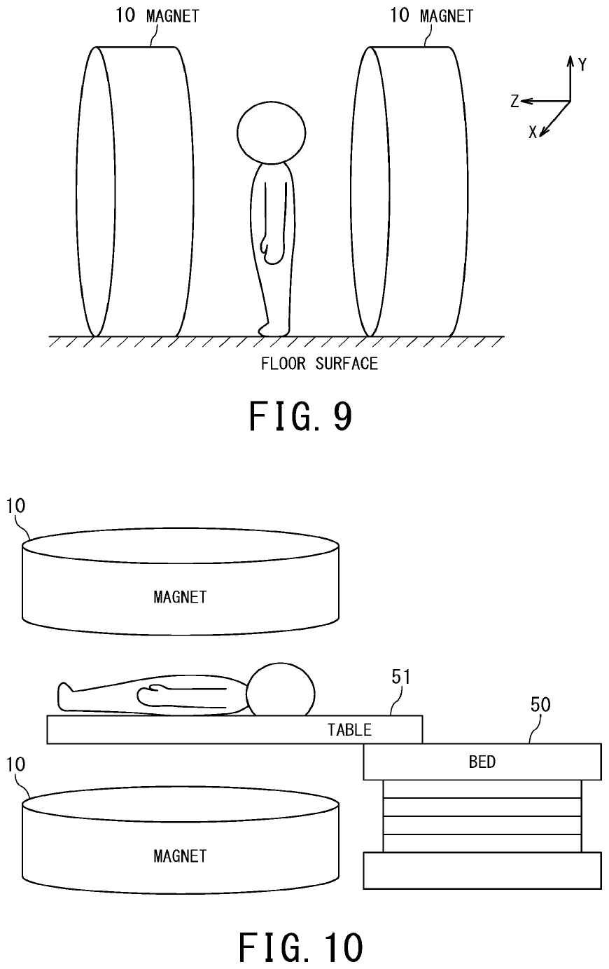

FIG. 9 is a schematic diagram illustrating a first configuration of the superconducting magnet 10 according to the second embodiment. In the second embodiment as shown in FIG. 9, for example, two superconducting magnets 10 having a circular plate shape (i.e., thin cylindrical shape) are provided.

Each superconducting magnet 10 is arranged such that the central axis (i.e., the axis passing through the centers of both circular end faces of the cylindrical shape) is parallel to the floor surface, for example. Further, the two superconducting magnets 10 are arranged so as to sandwich the object. Such arrangement generates a magnetic field in the free space between the two superconducting magnets 10. The object is imaged in this open space in a standing position, for example.

FIG. 10 is a schematic diagram illustrating a second configuration of the superconducting magnet 10 according to the second embodiment. While FIG. 9 shows a configuration for imaging the object in a standing position, FIG. 10 shows a configuration for imaging the object in a recumbent position lying on the table 51 extending from the bed body 50. When imaging the object in the recumbent position, the two superconducting magnets 10 are arranged such that their central axes are in the vertical direction as shown in FIG. 10. For example, one superconducting magnet 10 is disposed below the table 51 and the other superconducting magnet 10 is disposed above the table 51.

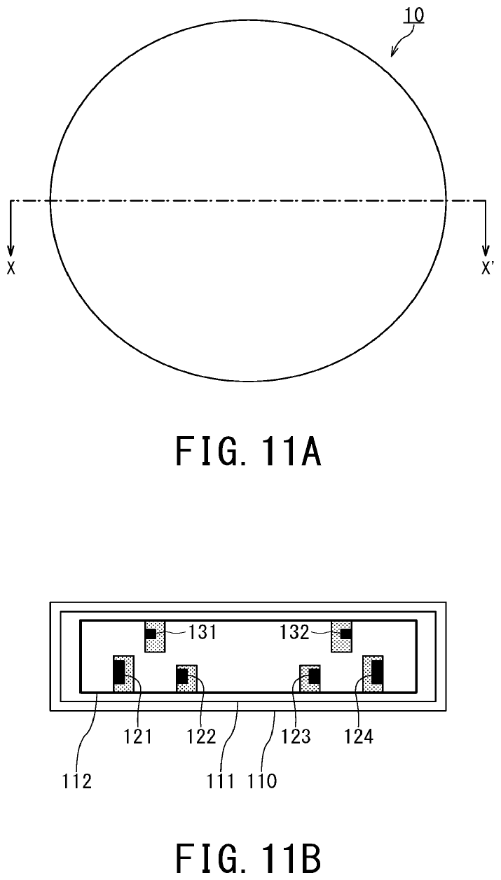

FIG. 11A is a plan view of one of the superconducting magnets 10, for example, the lower superconducting magnet 10 as viewed from above. FIG. 11B is a cross-sectional view of the superconducting magnet 10 taken along the line X-X′ in FIG. 11A for illustrating its internal configuration.

The superconducting magnet 10 of the second embodiment has substantially the same configuration as the superconducting magnet 10 of the first embodiment. Since the operation of the superconducting magnet 10 of the second embodiment is substantially the same as that of the first embodiment, duplicate description is omitted.

As shown in FIG. 9 to FIG. 11B, in the imaging using the superconducting magnet 10 of the second embodiment, the object can be imaged in an open magnetic field space, and thus, even a patient with claustrophobia can be imaged, for example.

Although FIG. 9 and FIG. 10 show a configuration including two superconducting magnets 10 facing each other, it can be a configuration including only one superconducting magnet 10. For example, a configuration including only one superconducting magnet 10 below the table 51 in FIG. 10 may be used.

As described above, the superconducting magnet of at least one embodiment can instantaneously increase or decrease the static magnetic field strength generated by the superconducting coil, while suppressing the risk of the occurrence of quench or unnecessary high voltage.

While certain embodiments have been described, these embodiments have been presented by way of example only, and are not intended to limit the scope of the invention. Indeed, the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms or modifications as would fall within the scope and spirit of the invention.

What is claimed is:

1. A superconducting magnet comprising:
at least one primary superconducting coil configured to generate a primary static magnetic field by a persistent current flowing during a persistent current mode;
at least one secondary superconducting coil configured to generate a secondary static magnetic field different from the primary static magnetic field in response to external control; and
a static-magnetic-field control switch configured to
supply the secondary superconducting coil with part of the persistent current to generate the secondary static magnetic field by being closed in response to the external control during the persistent current mode and
stop energization of the secondary superconducting coil and generation of the secondary static magnetic field by being opened in response to the external control during the persistent current mode.

2. The superconducting magnet according to claim 1, wherein:
the static-magnetic-field control switch comprises a superconducting member and a heater configured to heat the superconducting member;
the static-magnetic-field control switch is opened when stopping generation of the secondary static magnetic field by turning on the heater under the external control and bringing the superconducting member into a normal conducting state; and
the static-magnetic-field control switch is closed when generating the secondary static magnetic field by turning off the heater under the external control and shifting the superconducting member from the normal conducting state to a superconducting state.

3. The superconducting magnet according to claim 2, wherein, the superconducting member provided in the static magnetic field control switch is configured by non-inductively winding a superconducting wire.

4. The superconducting magnet according to claim 1, further comprising a persistent current switch configured to:
be opened during an excitation mode to allow an electric current supplied from an external power supply to flow through the primary superconducting coil; and
be closed during the persistent current mode to form a persistent current loop with the primary superconducting coil in such a manner that the persistent current flows through the primary superconducting coil.

5. The superconducting magnet according to claim 4, wherein:
the persistent current switch comprises a superconducting member and a heater configured to heat the superconducting member;
the persistent current switch is opened during the excitation mode by turning on the heater under the external control and bringing the superconducting member into a normal conducting state; and
the persistent current switch is closed during the persistent current mode by turning off the heater under the external control and shifting the superconducting member from the normal conducting state to a superconducting state.

6. The superconducting magnet according to claim 1, wherein the secondary superconducting coil is configured to generate the secondary static magnetic field in such a manner that the primary static magnetic field is canceled or reduced.

7. The superconducting magnet according to claim 1, wherein the secondary superconducting coil is configured to generate the secondary static magnetic field due to an emergency magnetic-field shutdown function in such a manner that the primary static magnetic field is canceled by the secondary static magnetic field, and strength of a combined magnetic field of primary and secondary static magnetic fields is instantaneously reduced from a rated value to zero without bringing the primary superconducting coil into a quenched state.

8. The superconducting magnet according to claim 1, wherein the secondary superconducting coil is configured to generate the secondary static magnetic field in such a manner that spatial distribution of a combined static magnetic field obtained by combining the primary static magnetic field and the secondary static magnetic field changes.

9. The superconducting magnet according to claim 1, wherein the secondary superconducting coil is configured as a coil that is non-inductively wound with respect to the primary superconducting coil.

10. The superconducting magnet according to claim 1, wherein the external control is performed in synchronization with a pulse sequence in magnetic resonance imaging.

11. The superconducting magnet according to claim 1, wherein:
the static-magnetic-field control switch comprises a superconducting member and a heater configured to heat the superconducting member;
the external control is performed in synchronization with a pulse sequence in magnetic resonance imaging; and
a timing of the external control is determined in consideration of a delay time from turning on or off the heater until the static-magnetic-field control switch closes or opens, with respect to a change timing of the primary static magnetic field in the pulse sequence, or a combined static magnetic field obtained by combining the primary static magnetic field and the secondary static magnetic field.

12. The superconducting magnet according to claim 1, wherein a resistor or a diode is provided in parallel with the secondary superconducting coil.

13. An MRI apparatus comprising the superconducting magnet according to claim 1.
